(19) United States
(12) Patent Application Publication
(10) Pub. No.: US 2011/0251648 A1
(43) Pub. Date: Oct. 13, 2011

(54) IMPLANT SYSTEM FOR STABILIZING BONES

(76) Inventors: Meinrad Fiechter, Munsingen (CH); Friedrich Magerl, Gallen (CH); Ronald Wieling, Eichberg (CH)

(21) Appl. No.: 13/141,037

(22) PCT Filed: Dec. 8, 2009
(86) PCT No.: PCT/EP2009/008742

§ 371 Date: Jun. 20, 2011

(30) Foreign Application Priority Data
Dec. 19, 2008 (EP) 08 405 310.7

(51) Int. Cl. A61B 17/80; A61B 17/84

(52) U.S. Cl. 606/286; 606/300

(57) ABSTRACT

The invention relates to an implant system having at least one component (6, 24, 33, 39) comprising an elongated body (15, 25, 34, 40) having at least one segment at which it can be connected to an attachment system (2, 3; 42) of the implant system, such as a pedicle screw or a brace. The elongated body (15, 25, 34, 40) is made of a plastic transparent to X-rays and is fixedly connected to a substantially harder part (16, 17, 27, 28, 35, 37, 41) in the at least one segment named above, said harder part forming an interface to said attachment element (2, 42).

The invention relates to an implant system for stabilizing bones, with a component having an elongate body which has at least one segment at which it can be connected to another part, for example to a pedicle screw or a clip.

Implant systems of this kind are used, for example, for stabilizing segments of the spinal column. These systems comprise an elongate body, which is designed as a rod or plate and which is anchored in vertebral bodies with at least two pedicle screws. Implant systems of this kind are known in numerous designs in the prior art and are disclosed in U.S. Pat. No. 5,474,555 and EP-A-0 746 255, for example. In these systems, said rods each form structural components and the pedicle screws form securing elements. The rods are made, for example, of titanium, implant steel or another biocompatible metal. These have the disadvantages of a lack of transparency to X-rays and the formation of artifacts. Moreover, the modulus of elasticity is too high for some uses. Rods and plates made of plastic, in particular PEEK and carbon-fiber-reinforced PEEK, are also known. These are transparent to X-rays and do not generate artifacts. Moreover, they generally have a modulus of elasticity close to that of bone. However, the strength and hardness are in many cases too low. Another disadvantage is the abrasion at the securing segments, which can include parts of fibers or of the matrix.

WO 2006/118866 has disclosed a fixation system that has a rod made of metal and plastic. The rod is intended to afford the advantage that its flexural strength and its modulus of elasticity can be varied by corresponding different configuration of the two materials. Here too, however, a disadvantage is the lack of transparency to X-rays.

Implant systems are also known that have an intramedullary nail, for example an intramedullar hip nail. This likewise forms an elongate component which, for example, has to be connected to a femoral neck screw. These systems also have the above-mentioned disadvantages, particularly the lack of transparency to X-rays and the formation of artifacts. If the intramedullary nail is a steel nail, there is also the disadvantage that the modulus of elasticity thereof is substantially greater than the bone that is to be stabilized.

The object of the invention is to make available an implant system that is of said type and that avoids the disadvantages mentioned.

In an implant system of the type in question, the object is achieved by the fact that the elongate body is made of a plastic transparent to X-rays and is fixedly connected, in said at least one segment, to a further part, which further part forms an interface to said other part. In the implant system according to the invention, a component is therefore used in which the only areas not transparent to X-rays are the segments where the component is connected via a pin to, for example, a securing element, a pedicle screw, a femoral neck screw or a fixator. The component can be clamped, for example, with comparatively great force at these segments or interfaces. The clamping takes place on the substantially harder part. Abrasion of the plastic can thus be avoided. Since the component is made of plastic outside the securing segments, it is largely transparent to X-rays and the formation of artifacts can be substantially reduced. Outside the securing segments, the component also has a modulus of elasticity corresponding to that of the bone to be stabilized.

According to a development of the invention, the harder part is arranged on at least one end of the elongate body. In particular, the plastic transparent to X-rays has a modulus of elasticity that is closer to the modulus of elasticity of bone than is the harder part. A particularly stable securing of the securing element on the component is possible when the further part is made of metal, in particular titanium, a titanium alloy, implant steel or plastic. This harder part can be comparatively short. According to a development of the invention, it is sleeve-shaped. The sleeve is fixedly connected to the elongate body made of plastic. The connection can, for example, be an interference fit or press fit. A connection is also possible by means of a thread or by a welded connection. Moreover, the part made of plastic can be injected onto the harder part.

A particularly stable connection is ensured if, according to a development of the invention, the further part is structured on the outside for a form-fit connection to the securing element. With such a form-fit connection, a radial and/or axial relative movement in particular can be avoided. The structuring can in particular be provided by a plurality of depressions or elevations. The securing element preferably has corresponding depressions or elevations.

Further advantageous features will become clear from the dependent claims, from the description below, and from the drawing.

Illustrative embodiments of the invention are explained in more detail below with reference to the drawing, in which:

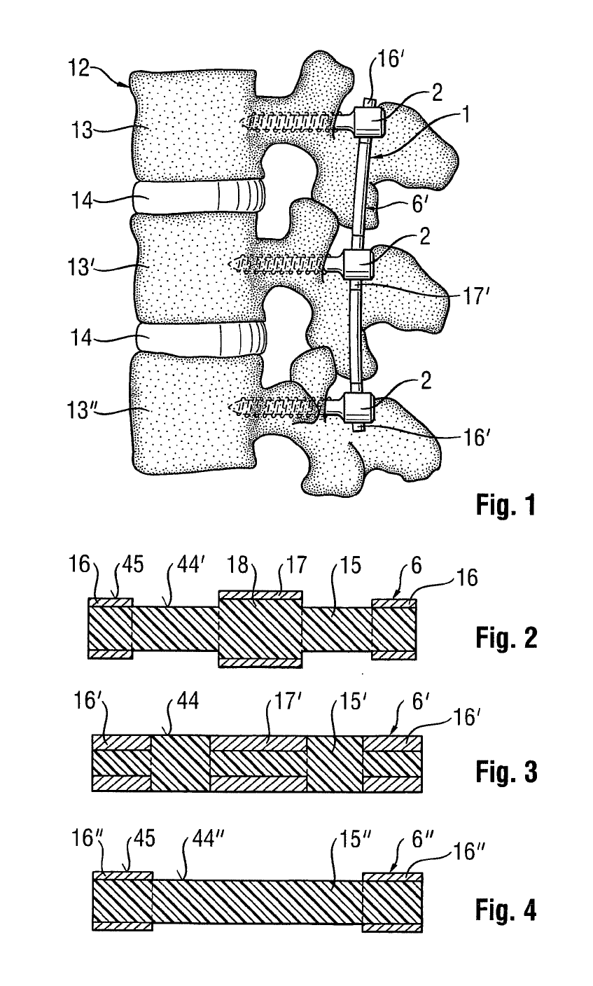

FIG. 1 shows a schematic view of an implant system according to the invention for stabilizing a segment of a spinal column,

FIG. 2 shows a section through a rod of the implant system according to FIG. 1,

FIG. 3 shows a section through a rod according to one variant,

FIG. 4 shows a section through a rod according to a further variant,

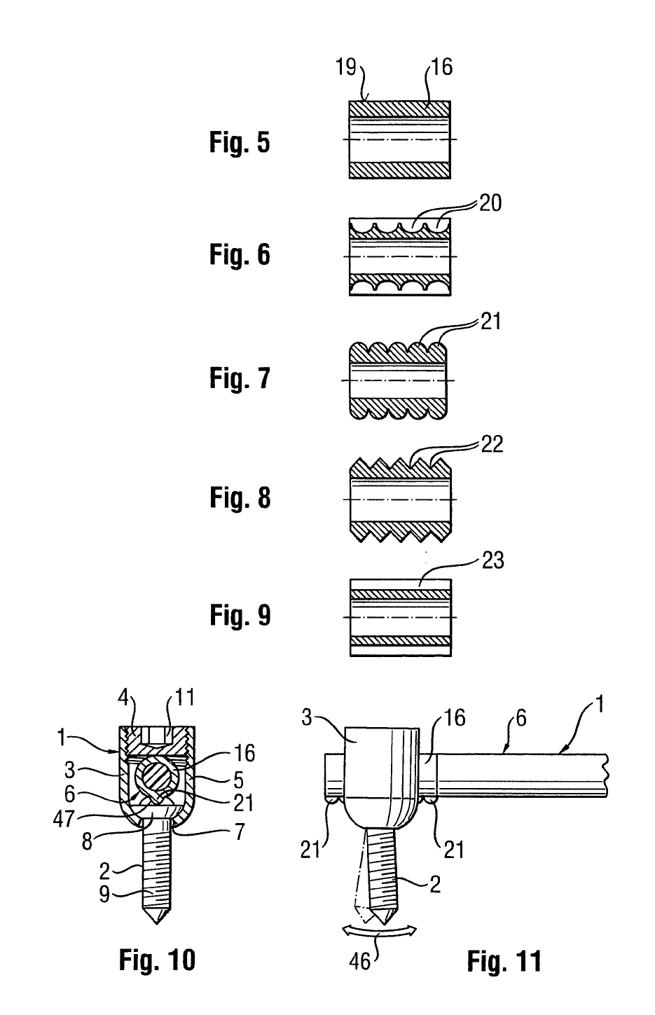

FIGS. 5-9 show sections through sleeves according to different variants,

FIG. 10 shows a schematic view of a section through an anchor that has a pedicle screw and that secures a rod on a vertebral body,

FIG. 11 shows a view of an anchor and a segment of a rod,

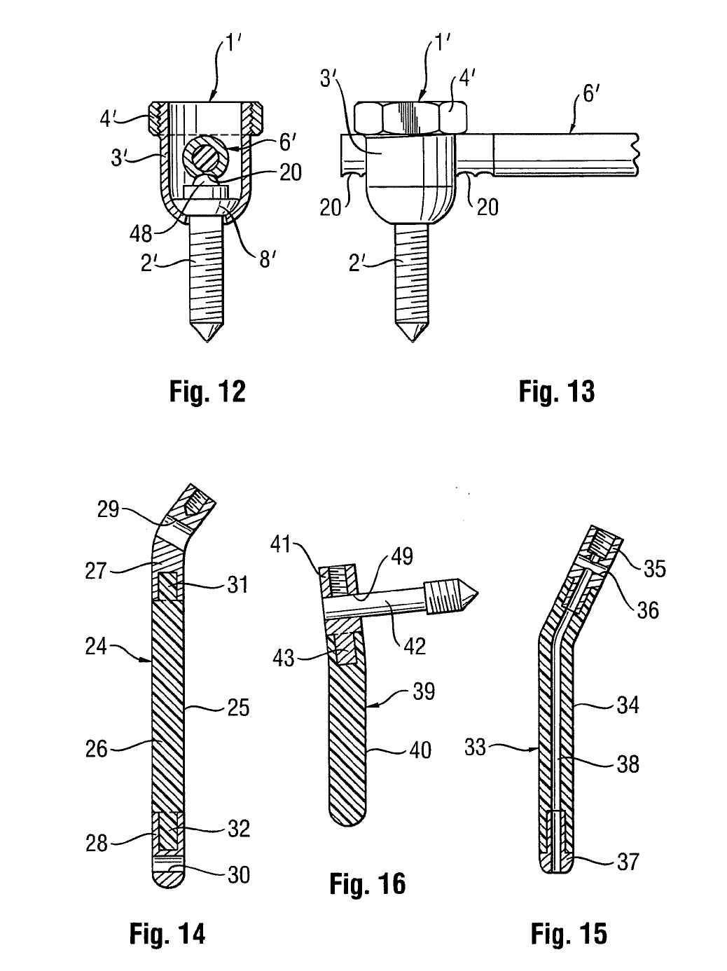

FIG. 12 shows a section through an anchor and a rod according to one variant,

FIG. 13 shows a view of the anchor and of a segment of the rod according to FIG. 12,

FIG. 14 shows a section through an intramedullary nail,

FIG. 15 shows a section through an intramedullary nail according to one variant,

FIG. 16 shows a section through an intramedullary hip nail,

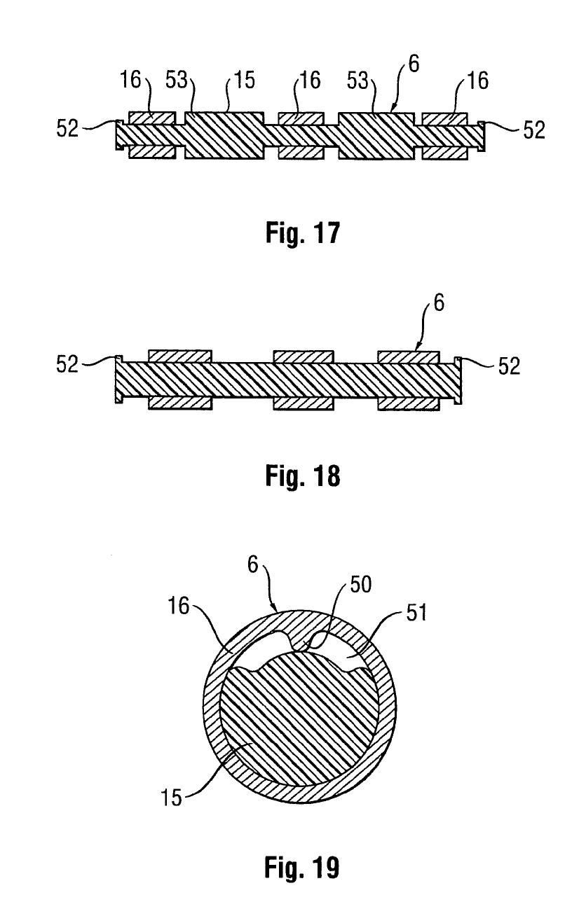

FIG. 17 shows a longitudinal section through a rod according to a further variant,

FIG. 18 shows a longitudinal section through a rod according to a further variant, and

FIG. 19 shows a cross section through a rod according to a further variant.

FIG. 1 shows a stabilizing arrangement 1 or an implant system with which three vertebrae 13, 13′ and 13″ of a spinal column 12 are stabilized. The intervertebral disks 14, in which intervertebral elements not shown here can be inserted, are located between the vertebrae 13, 13′ and 13″. The stabilizing arrangement 1 is intended in particular to permit a fusion of the vertebrae 13, 13′ and 13″. The stabilizing arrangement 1 comprises three pedicle screws 2, which can be of identical design, and a connecting rod 6′, which can be straight or bent. The stabilizing arrangement 1 or the implant system generally comprises a further connecting rod 6′, which is concealed here and is likewise anchored with three pedicle screws 2. The connecting rods 6′ are components of the stabilizing arrangement 1 and connect the three vertebrae 13, 13′ and 13″ to one another.

The connecting rod 6′ shown in FIG. 3 is composed of an elongate body 15′, which is made of a comparatively light material, in particular of plastic. The plastic is, for example, PEEK or carbon-fiber-reinforced PEEK, PEK or a similar material. This elongate body 15′ is fixedly connected to two outer sleeves 16′ and to a middle sleeve 17′. These sleeves 16′ and 17′ are made of a material which, for example, is substantially harder than the material of the body 15′. On the outside, the sleeves 16′ and 17′ are flush with an outer face 44 of the connecting rod 6′. The connecting rod 6′ is preferably circular in cross section, although another cross section is also possible, for example an oval or polygonal cross section. The sleeves 16′ and 17′ are made in particular of titanium, a titanium alloy or an implant steel. They form the interface or the securing segments at which the connecting rod 6′ is connected to clamping devices 3.

The connection is in particular a clamped connection, preferably a form-fit clamped connection. Outside these segments, the body 15′, as can be seen, forms the outer face. Between the sleeves 16′ and 17′, the connecting rod 6′ is therefore transparent to X-rays. Since the connecting rod 6′ is secured on the sleeves 16′ and 17′, the body 15′ is not appreciably subjected to clamping forces, for example. In particular, undesired abrasion particles cannot be produced, for example fibers or a matrix of the body 15′. Such abrasion particles can lead to undesired reactions.

The connecting rod 6 shown in FIG. 2 differs from the one according to FIG. 3 mainly in that the body 15 has an outer face 44′, which is recessed in relation to at least one outer face 45 of a sleeve 16. Moreover, the connecting rod 1 is provided, approximately at the center, with a thickened area 18 on which a sleeve 17 is secured. The external diameter of the sleeve 17 is also greater than the diameter of the sleeves 16.

FIG. 4, finally, shows a connecting rod 16″ in which only two sleeves 16″, arranged at the ends, are provided on a body 15″. This connecting rod 6″ is connected only to two pedicle screws 2. Between the sleeves 16″, there is therefore in this case a relatively large area in which the connecting rod 6″ is transparent to X-rays. In this area, the modulus of elasticity corresponds to that of the body 15″. However, embodiments are also conceivable here in which more than three sleeves 16″ are provided. In the connecting rod 6″ according to FIG. 4, the outer face 44″ of the body 15″ can be flush with the outer face 45″ of the sleeves 16″.

The sleeves 16 and 17 are preferably cylindrical, although they can also have a conical shape. The connection is a fixed connection, that is to say the sleeves 16 and 17 are connected permanently to the body 15. The connection can be, for example, an adhesively bonded connection, a welded connection, or a connection by form-fit engagement. Production in an injection molding machine is possible in particular. The sleeves 16 and 17 are in this case inserts in the die. The body 15 is then injected onto these inserts.

The sleeves 16 and 17 are preferably structured on the outside. However, a sleeve 16 is also conceivable which, according to FIG. 5, has a smooth outer face 19. The cross section of this sleeve 16 can be oval or polygonal, for example. In the embodiment according to FIG. 6, the outer face is structured by a plurality of depressions 20. These depressions 20 can be hemispherical, for example. However, the structuring can also be formed by elevations 21 according to FIG. 7, in which case these elevations can likewise be hemispherical, for example. In the embodiment according to FIG. 8, depressions 22 are provided that are circumferential grooves. In the embodiment according to FIG. 9, grooves 23 are likewise provided, but these extend axially along the entire length of the sleeve 16. The sleeve 17 can be structured correspondingly. The sleeves 16 and 17 can be structured identically or differently. For example, an embodiment is conceivable in which one of the sleeves 16 or the sleeves 16 and the sleeve 17 are differently structured. One of these sleeves can, for example, have grooves 22 according to FIG. 8 and the other can have grooves 23 according to FIG. 9. The connecting rod 6 is then fixed both axially and radially. The structuring preferably extends over the entire outer face of the sleeve 16, 17. However, an embodiment is also conceivable in which only a partial area of the outer face is structured.

FIGS. 10 and 11 show the form-fit connection of a connecting rod 6 to the clamping device 3. The latter has a sleeve-shaped support 5 with an opening 1 through which the shank 9 of the pedicle screw 2 extends. The pedicle screw 2 has a head 8, which is mounted in the support 5 in such a way that the pedicle screw 2 is movable polygonally in the unclamped state, as is indicated by arrow 46 in FIG. 11. However, the polygonal mobility is not essential. The connecting rod is pressed onto the head 8 by means of a clamping element 4, for example a nut with a hexagon socket 11. In the clamped state, the pedicle screw 2 is immovable with respect to the support 5 and also with respect to the connecting rod 6. The screw head 8 has on its circumference at least and preferably several depressions 47 designed corresponding to the elevations 21 of the sleeve 16 on which the connecting rod 6 is clamped. In the clamped state, the pedicle screw 2 is therefore connected to the connecting rod 6 by form-fit engagement. A form-fit connection is also conceivable between the clamping element 4 and the connecting rod 6. In this case, the clamping element 4 has at least one depression (not shown here) in which an elevation of the sleeve 16 engages. A still stronger connection is obtained in this way.

In the embodiment according to FIGS. 12 and 13, a head 8′ of a pedicle screw 2′ has an elevation 48, which engages in a depression 20 of a connecting rod 6′. Here too, a form-fit connection is accordingly provided between the pedicle screw 2′ and the connecting rod 6′. The clamping of the connecting rod 6′ is in this case effected by a nut 4′, which is screwed onto the outside of the clamping device 3′. FIG. 14 shows an intramedullary nail 24, which is likewise a component of an implant system and which has an elongate shaft 25 formed mainly by a body 26 of plastic. The body 26 can likewise be PEEK, PEK or another suitable plastic transparent to X-rays. The body 26 is fixedly connected by a pin 31 to a proximal element 27, which has a securing hole 29 at which the intramedullary nail 24 is connected to a customary securing element (not shown here). The proximal element 27 is likewise made of a material that is substantially harder than the material of the body 26. The material can likewise be titanium, a titanium alloy or an implant steel. The body 26 here has a second pin 32 via which a distal element 28 is connected to the body 26. The distal element 28 has a securing hole 30 for the engagement of a further securing element (not shown here). With the exception of the elements 27 and 28, the shaft 25 is therefore transparent to X-rays. The securing places only an inappreciable load on the body 26. A strong connection and in particular a clamped connection to said securing elements is nevertheless possible. This connection can, for example, be a screwed connection or clamped connection.

FIG. 15 likewise shows an intramedullary nail 33 with a shaft 34 on which are arranged a proximal element 35, with a securing hole 36, and a distal element 37. A passage 38 known per se runs through the shaft 34 and the elements 35 and 37. In the intramedullary nail 33 too, the shaft 34 between the elements 35 and 37 is made of a suitable plastic transparent to X-rays.

FIG. 16 shows a hip nail 39, which likewise forms an elongate component connected to a femoral neck screw 42. A shaft 40 is made of a plastic transparent to X-rays and is connected via a pin 43 to a distal element 41. This element 41 has a securing hole 49 at which the hip nail 39 is connected to the femoral neck screw 42. The distal element 41 at any rate is made of a material that is substantially harder than the material of the shaft 40. The advantages mentioned above are also achieved here. The materials here can be the same as those that have already been mentioned above with respect to the other illustrative embodiments. Markers known per se are conceivable in each case, for example barium sulfate, tantalum filaments or beads.

FIGS. 17, 18 and 19 show connecting rods 6 permitting an implant system, for example a pedicle system, that is partially dynamic. This dynamic effect can promote callus formation and can thus accelerate the formation of bone and, if appropriate, desired fusion.

In the embodiment according to FIG. 17, a body 15 is provided on which sleeves 16 are mounted with limited mobility in the longitudinal direction of the body 15. The sleeves 16 can be mounted on the body 15 so as to be able to rotate to a limited or unlimited extent in the circumferential direction of the latter. FIG. 19 shows how a sleeve 16 can be arranged to be able to rotate to a limited extent on a body 15. The sleeve 16 has, on an inner face, a knob 50 that engages in a recess 51 of the body 15. As can be seen, the rotation angle possible here is limited by abutment of the knob 50 on the body 15. The freedom of rotation could also be limited by other geometries of the sleeve 16 and of the body 15. For example, the body 15 and/or the sleeve 16 could have a polygonal design.

In the embodiment according to FIG. 17, the mobility of the sleeves 16 in the longitudinal direction is limited by outer abutments 52 and inner abutments 53.

In the embodiment according to FIG. 18, the inner abutments 53 are omitted. The outer abutments 52, however, are not essential and could also be omitted.

The abovementioned materials are possible for the sleeves 16 and the body 15. For example, the sleeves 16 can be made of titanium and the body 15 of plastic, for example PEEK.

### LIST OF REFERENCE SIGNS

1 stabilizing arrangement
2 pedicle screw
3 clamping device
4 clamping element
5 support
6 connecting rod
7 opening
8 screw head
9 shank
10 stabilizing arrangement
11 hexagon socket
12 spinal column
13 vertebral body
14 intervertebral disk
15 body
16 sleeve
17 sleeve
18 thickened area
19 outer face
20 depression
21 elevation
22 depression
23 groove
24 intramedullary nail
25 shaft
26 body
27 proximal element
28 distal element
29 securing hole
30 securing hole
31 pin
32 pin
33 intramedullary nail
34 shaft
35 proximal element
36 securing hole
37 distal element
38 passage
39 hip nail
40 shaft
41 distal element
42 femoral neck screw
43 pin
44 outer face
45 outer face
46 arrow
47 depression
48 elevation
49 securing hole
50 knob
51 recess
52 abutment
53 abutment

1-15. (canceled)

16. An implant system for stabilizing bones, said system comprising:
a component having an elongated body;
an other part; and
a further part forming an interface with said other part;
said elongated body comprising at least one segment at which said elongated body can be connected to said other part,
wherein said elongated body is made of a plastic material that is transparent to X-rays and is fixedly connected to said further part in said at least one segment.

17. The implant system as claimed in claim 16, wherein said further part is arranged on one end of said elongated body.

18. The implant system as claimed in claim 16, wherein said plastic material that is transparent to X-rays and said further part have respectively different moduli of elasticity.

19. The implant system as claimed in claim 16, wherein said further part is made of metal.

20. The implant system as claimed in claim 19, wherein said further part is made of a material selected from: titanium, a titanium alloy, an implant steel, and PEEK.

21. The implant system as claimed in claim 16, wherein said further part is sleeve-shaped.

22. The implant system as claimed in claim 16, wherein said further part has an outer structure configured to provide a form-fit connection to a securing element.

23. The implant system as claimed in claim 22, wherein the outer structure comprises a plurality of depressions or elevations.

24. The implant system as claimed in claim 16, wherein said elongated body has two ends and said further part comprises two further parts each arranged on a respective end of said elongated body.

25. The implant system as claimed in claim 16, wherein said component comprises one of: a connecting rod for connecting vertebral bodies; an intramedullary nail, and a connection element of an external fixator.

26. The implant system as claimed in claim 16, wherein said elongated body is made of a fiber-reinforced plastic.

27. The implant system as claimed in claim 26, wherein said fiber-reinforced plastic is fiber-reinforced PEEK.

28. The implant system as claimed in claim 16, wherein said component comprises a connecting rod, and said other part comprises a securing element in the form of an anchor that can be connected to a vertebral body and that has clamping means that can be pressed onto said further part.

29. The implant system as claimed in claim 28, wherein said further part has an outer structure, said clamping means has an area corresponding to the outer structure, and said outer structure and said area are configured to provide a form-fit connection between said further part and said clamping means.

30. The implant system as claimed in claim 16, wherein said component comprises an intramedullary nail having, at least at one of a distal end and a proximal end, a part that is harder than the remainder of said nail and in which a securing hole or another securing means is formed.

31. The implant system as claimed in claim 16, wherein said further part is movable with respect to said elongated body.

32. The implant system as claimed in claim 31, wherein said further part is mounted on said elongated body so as to be movable to a limited extent on said elongated body in at least one of an axial direction and a radial direction.

33. The implant system as claimed in claim 16, wherein said other part comprises a pedicle screw or a clip.
